(19) United States
(12) Patent Application Publication
(10) Pub. No.: US 2009/0285909 A1
(43) Pub. Date: Nov. 19, 2009

(54) Lactate and Calcium Containing Pharmaceutical Composition and Uses Thereof

(76) Inventor: Xavier M. Leverve, La Terrasse (FR)

Correspondence Address: ARNOLD & PORTER LLP;ATTN: IP DOCKETING DEPT., 555 TWELFTH STREET, N.W., WASHINGTON, DC, 20004-1206, US

(21) Appl. No.: 12/295,837

(22) PCT Filed: Apr. 3, 2006
(86) PCT No.: PCT/SG2006/000083

§ 371 Date: Jan. 28, 2009

(51) Int. Cl. A61K 33/00; A61P 9/00; A61P 25/00; A61K 33/06

(52) U.S. Cl. 424/682; 424/722

(57) ABSTRACT

The invention relates to a pharmaceutical composition containing 250 to 5000 millimoles per liter of lactate or lactic acid, 0.5 to 1.99 millimoles per liter of calcium and optionally 2 to 10 millimoles per liter of potassium. The invention also relates to pharmaceutical uses of this composition.

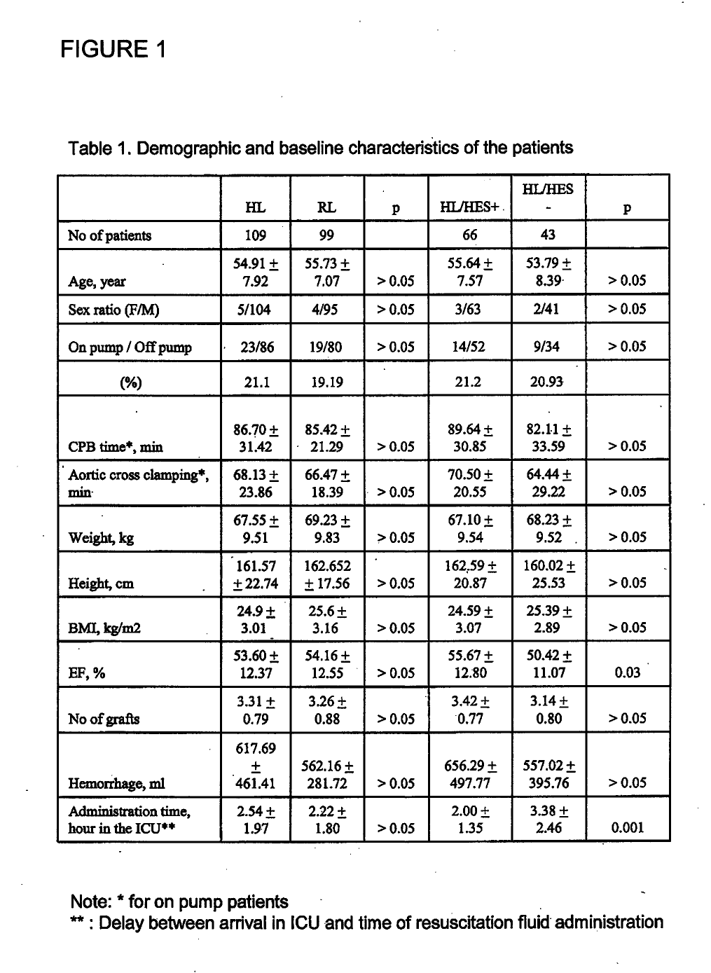

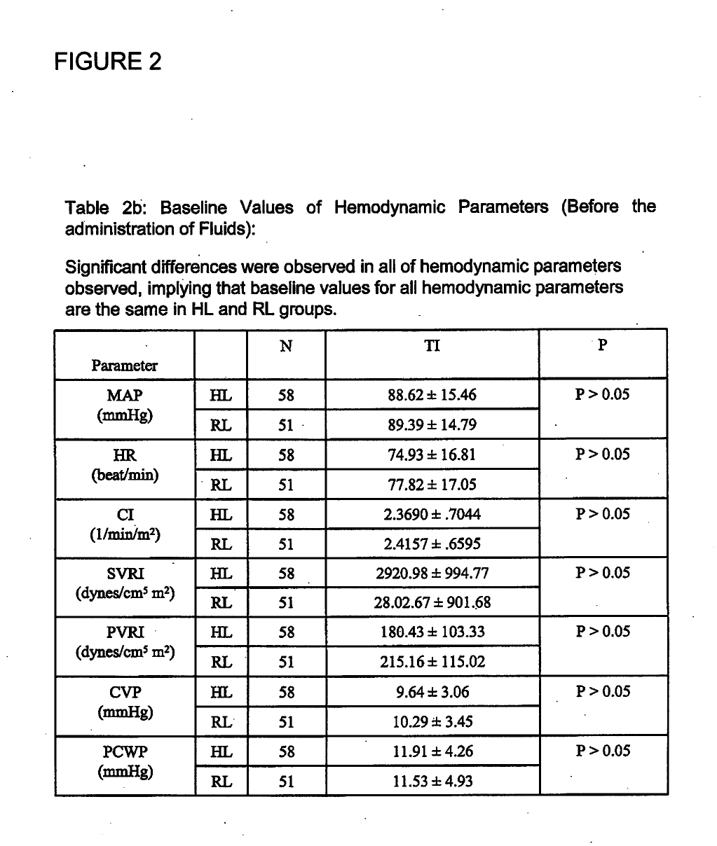

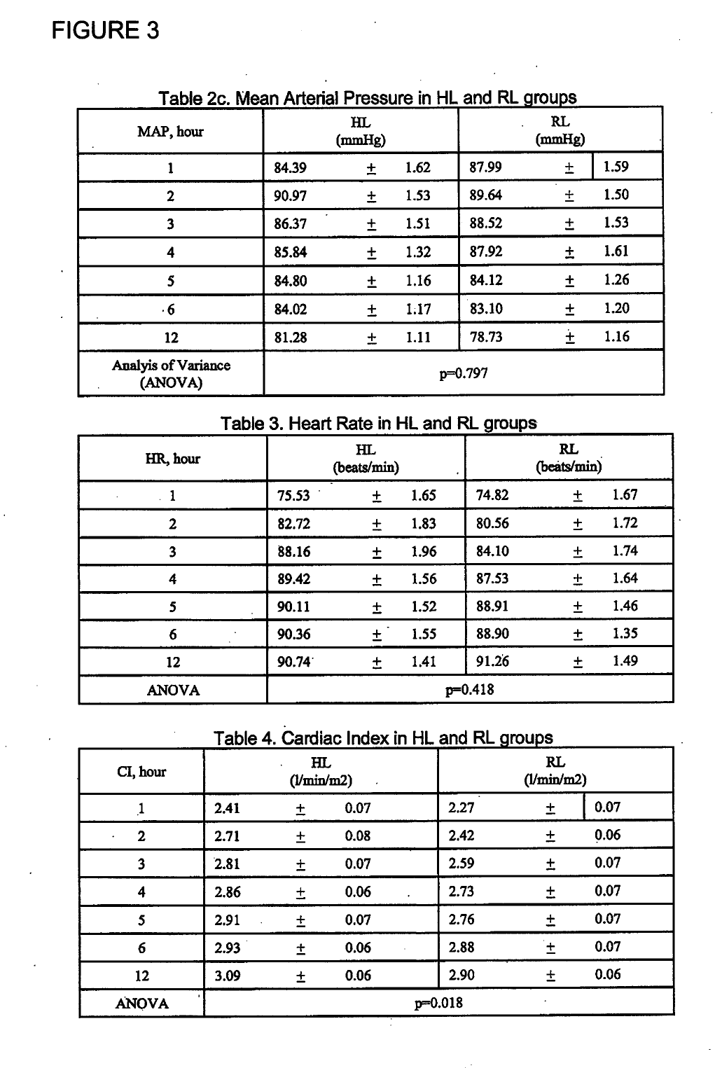

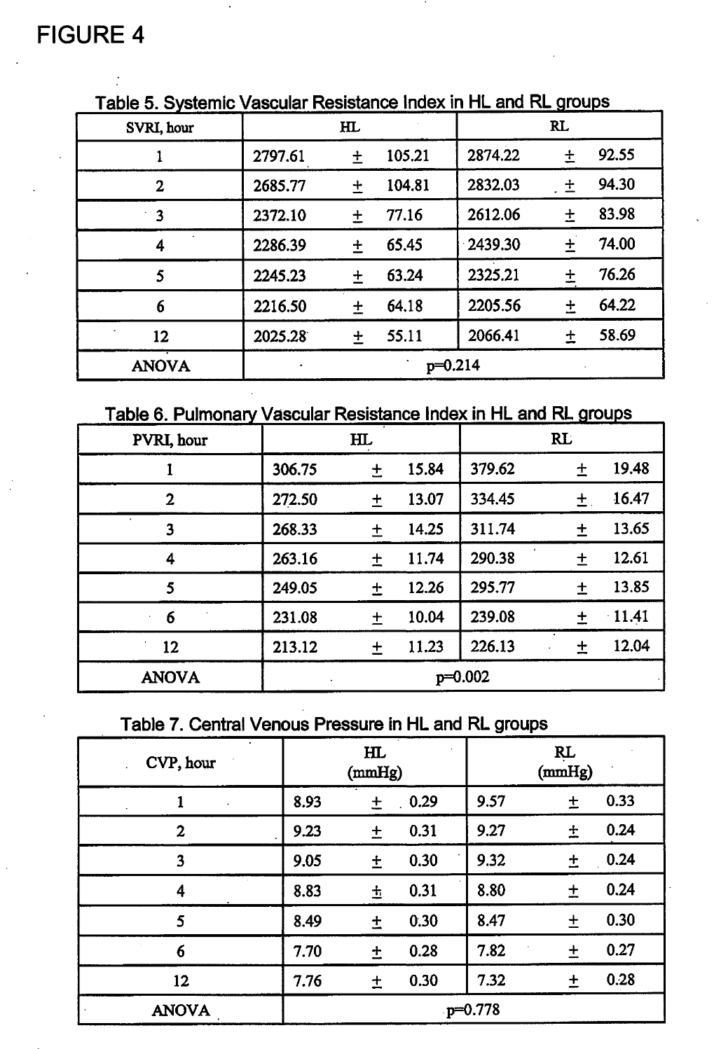

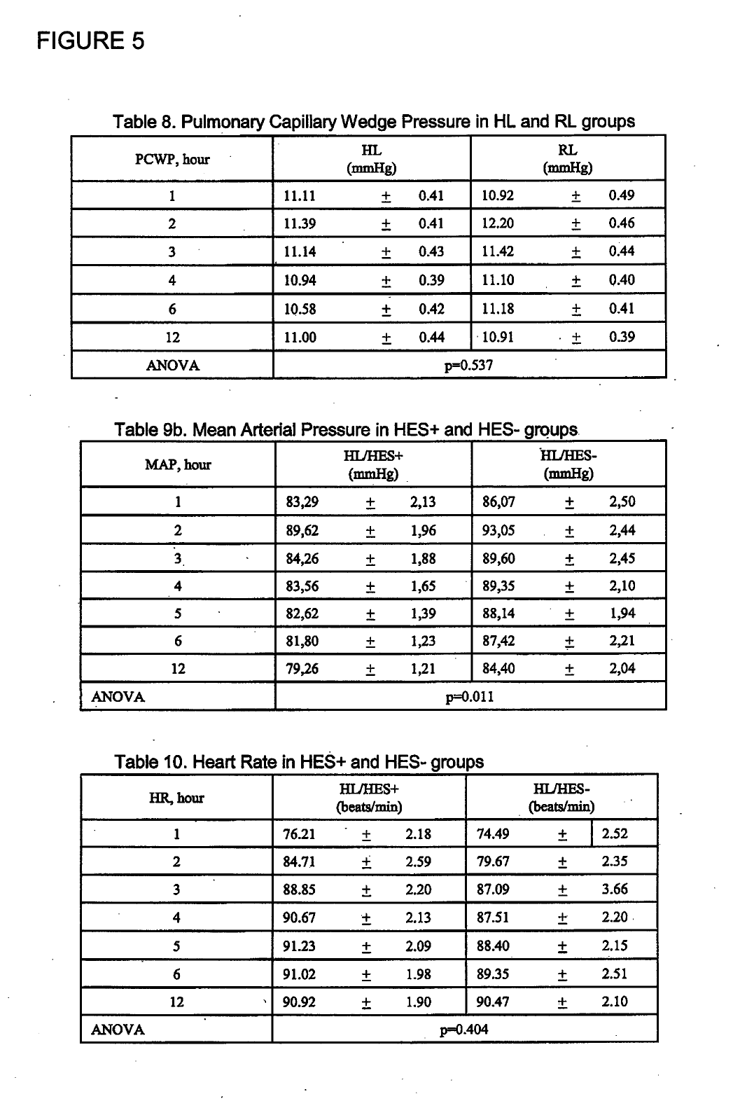

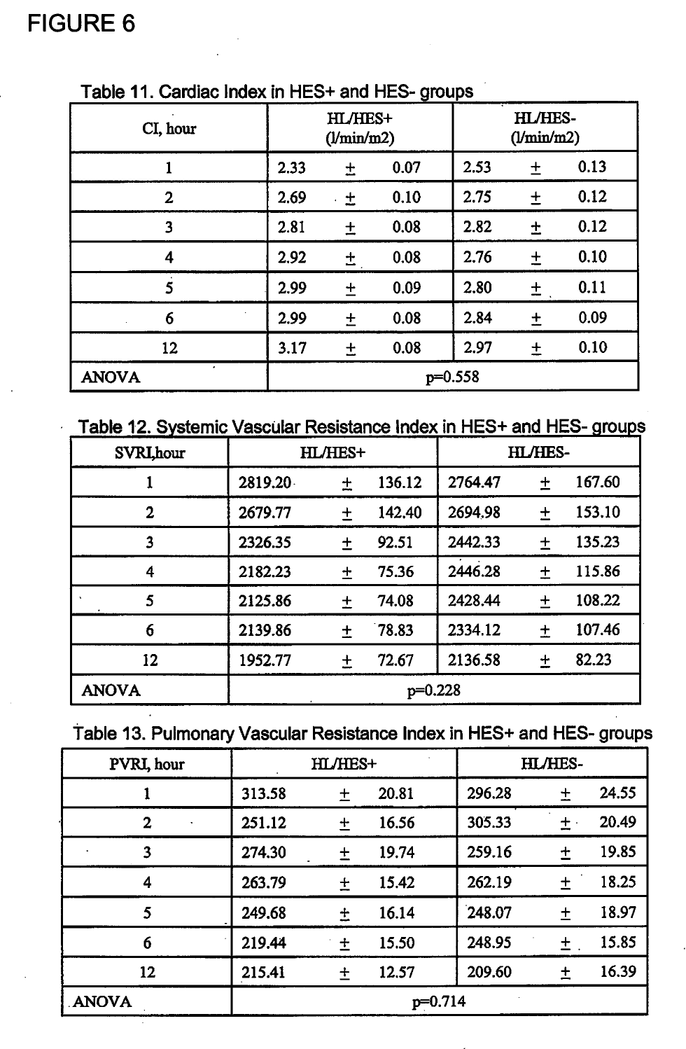

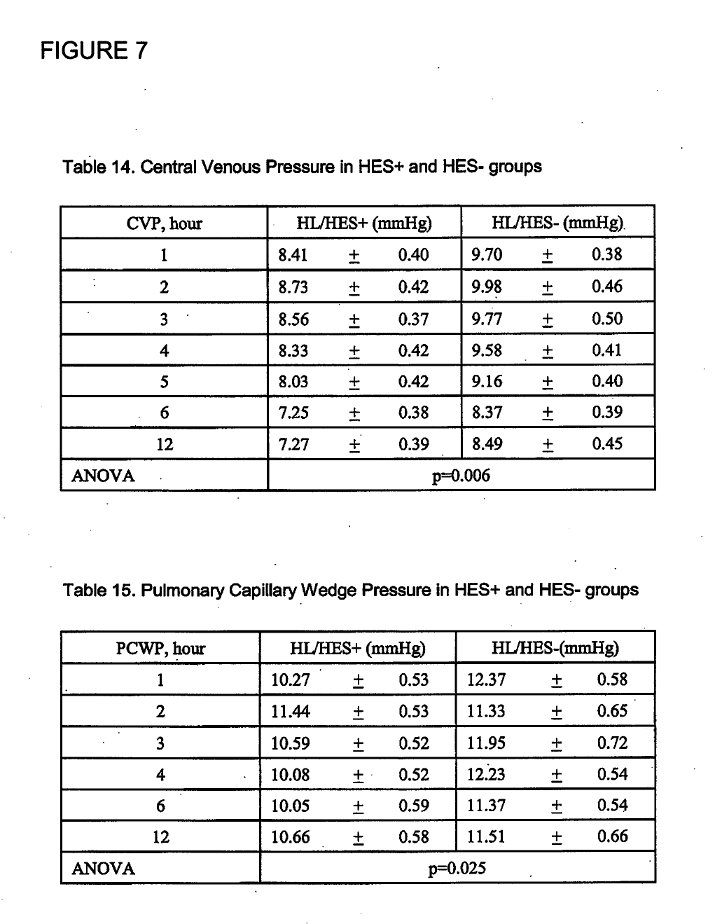

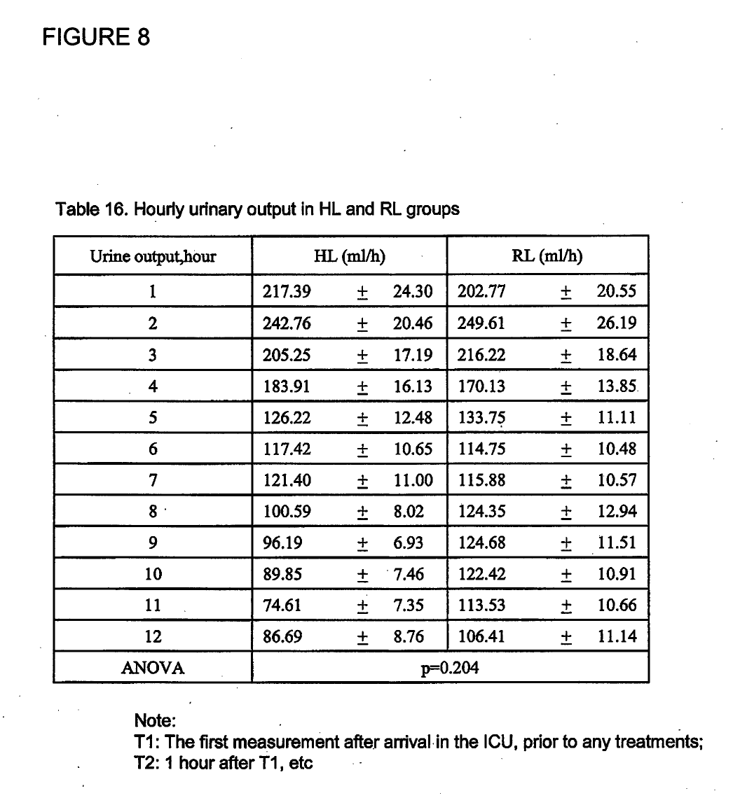

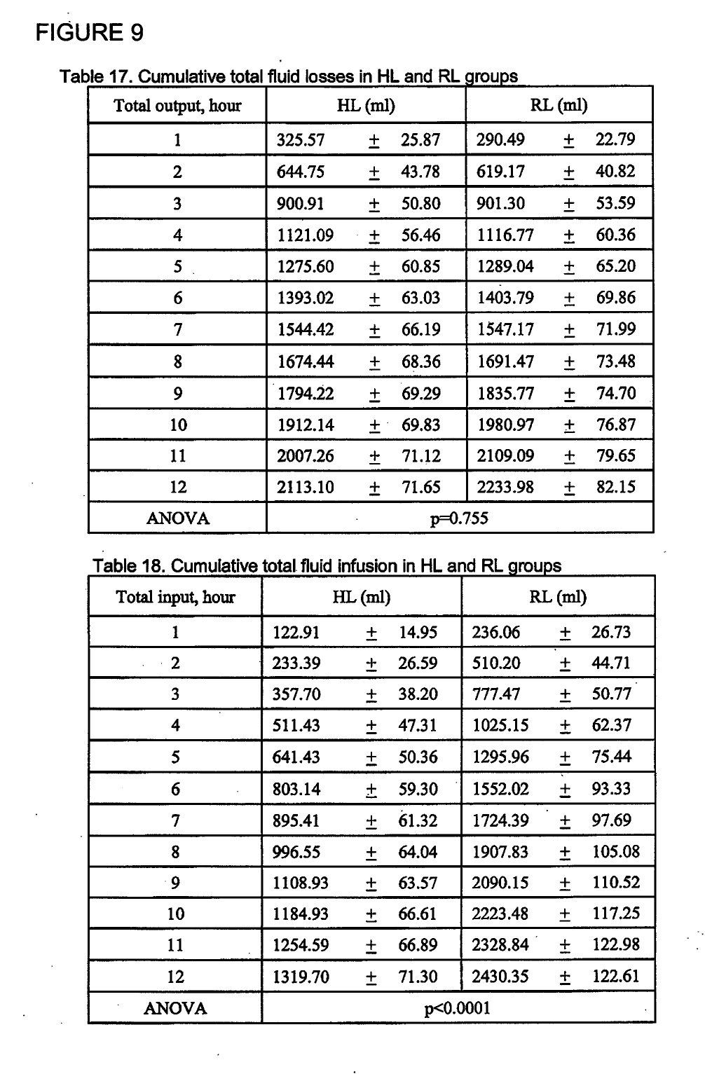

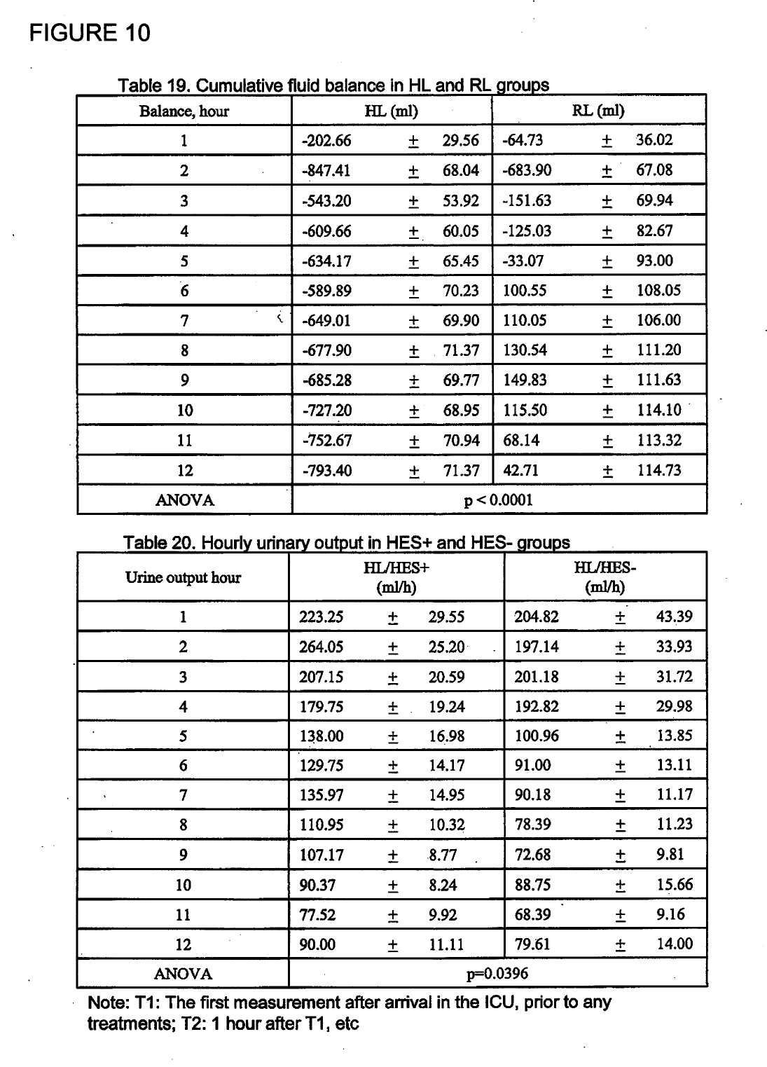

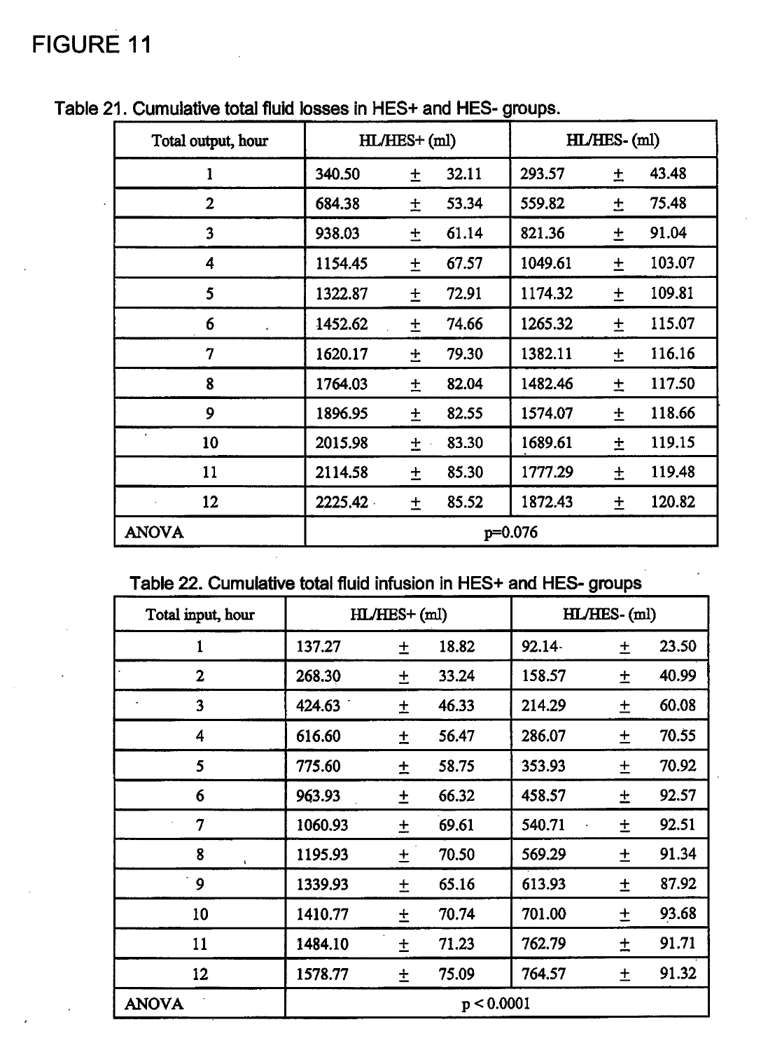

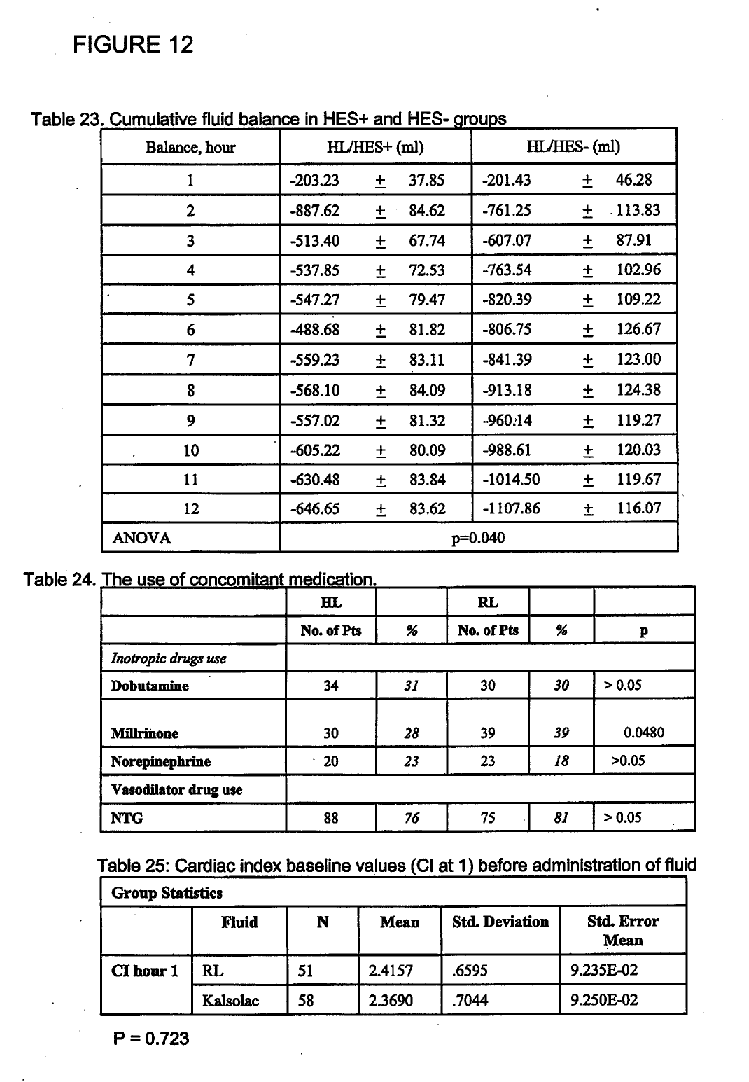

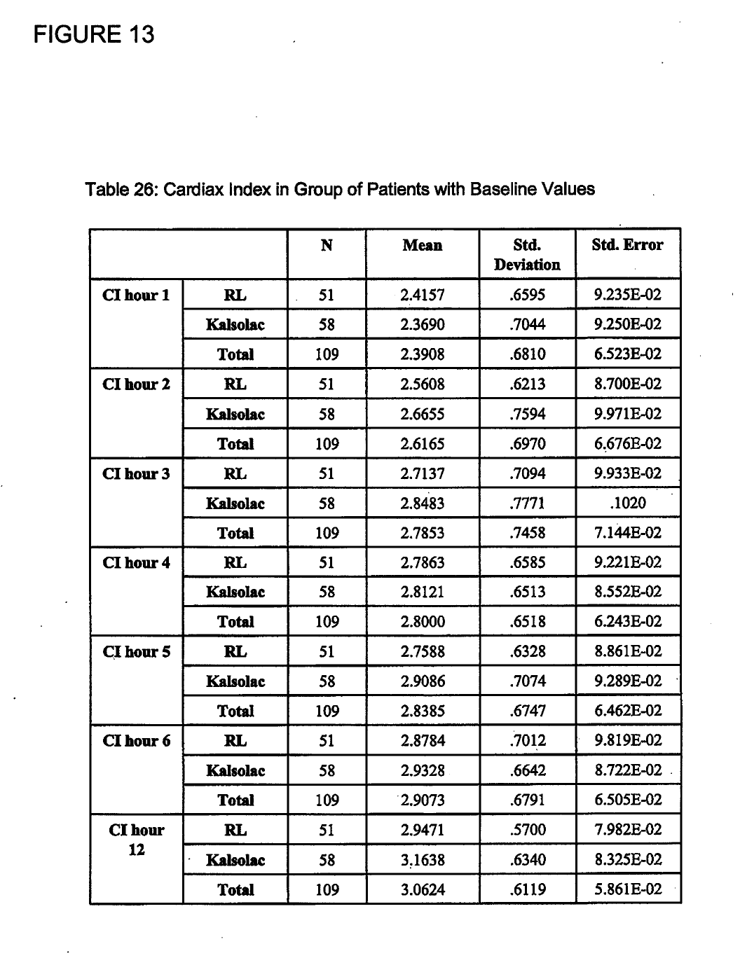

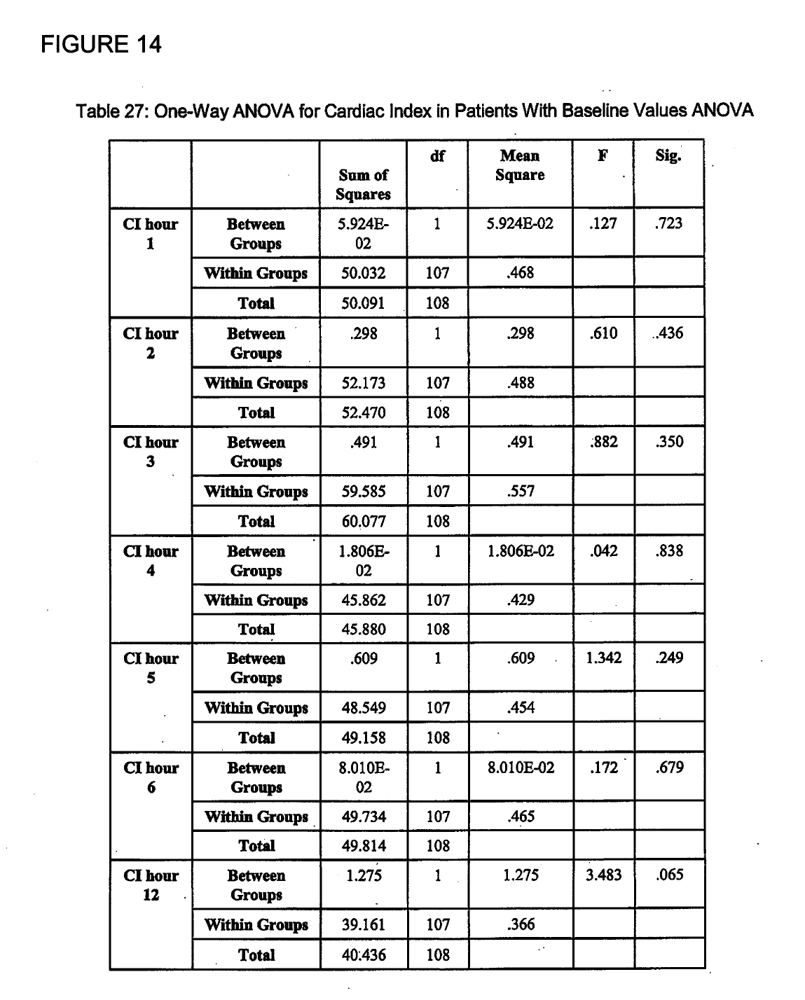

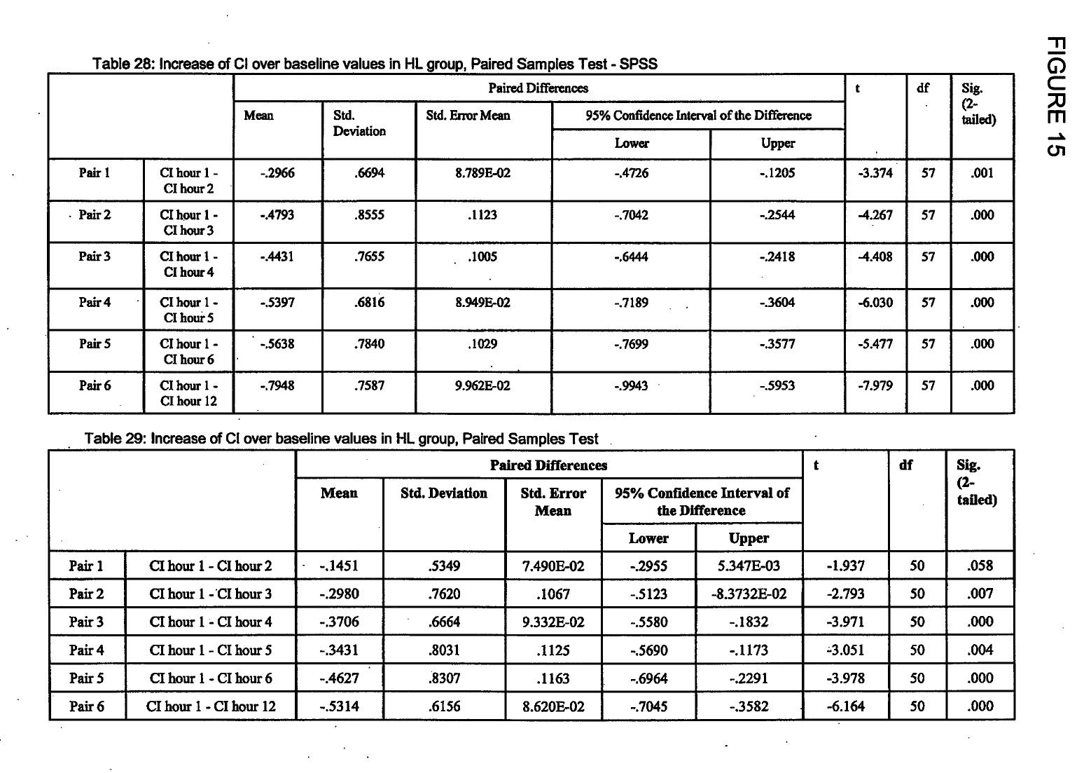

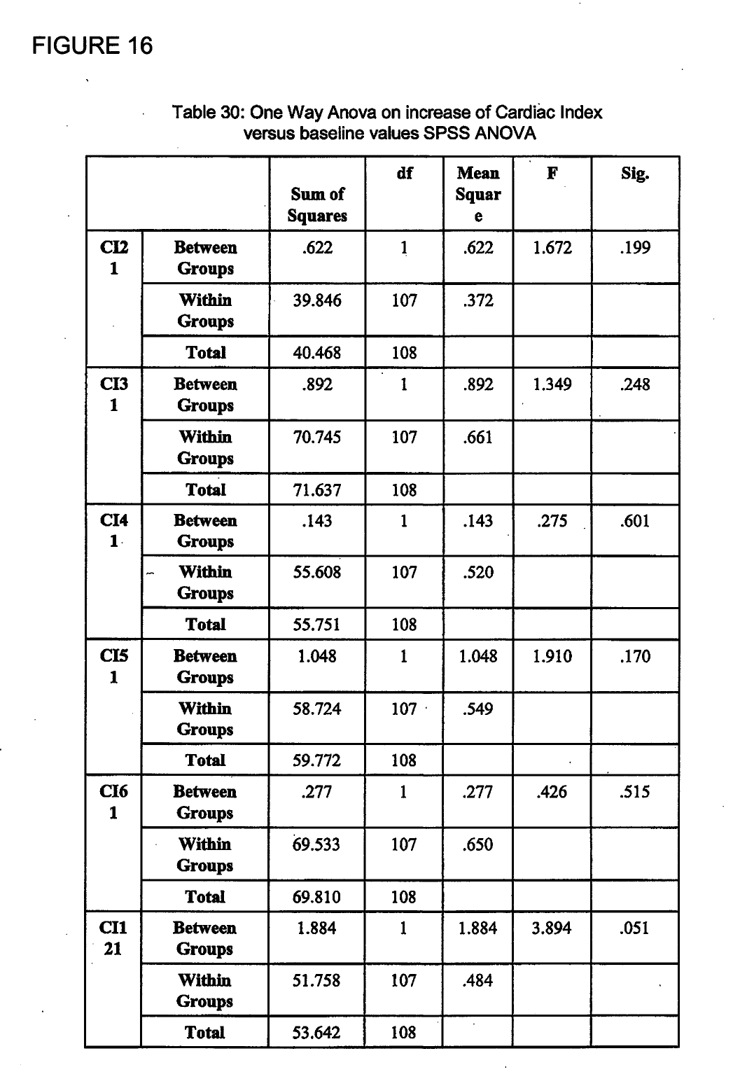

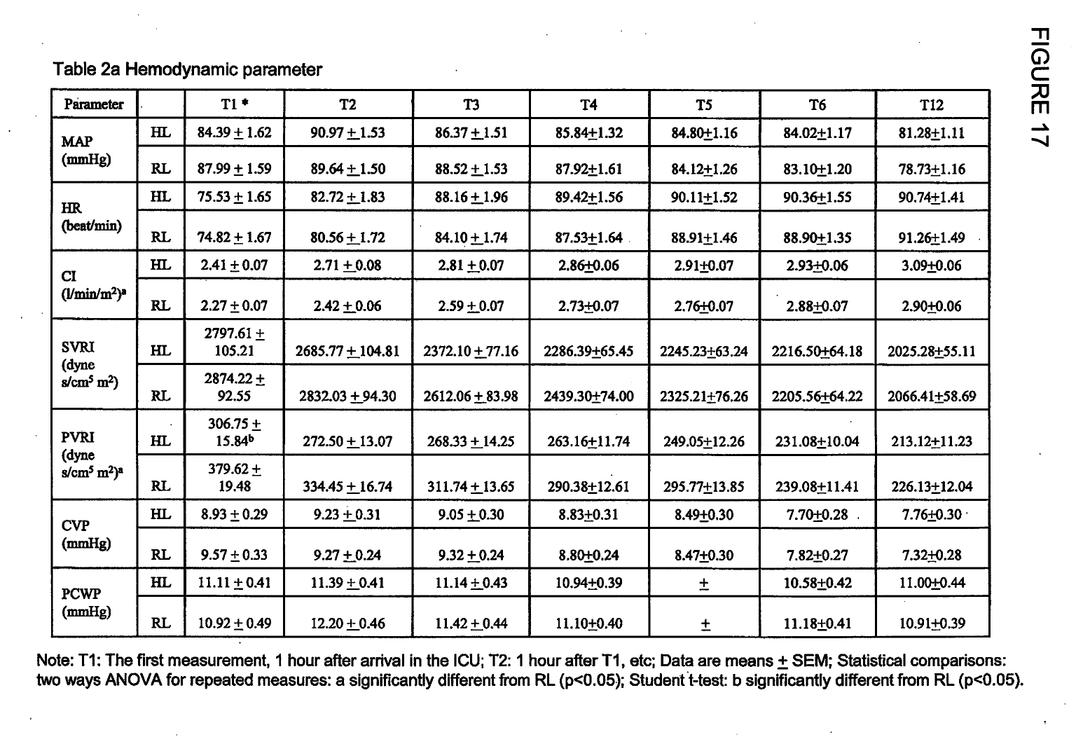

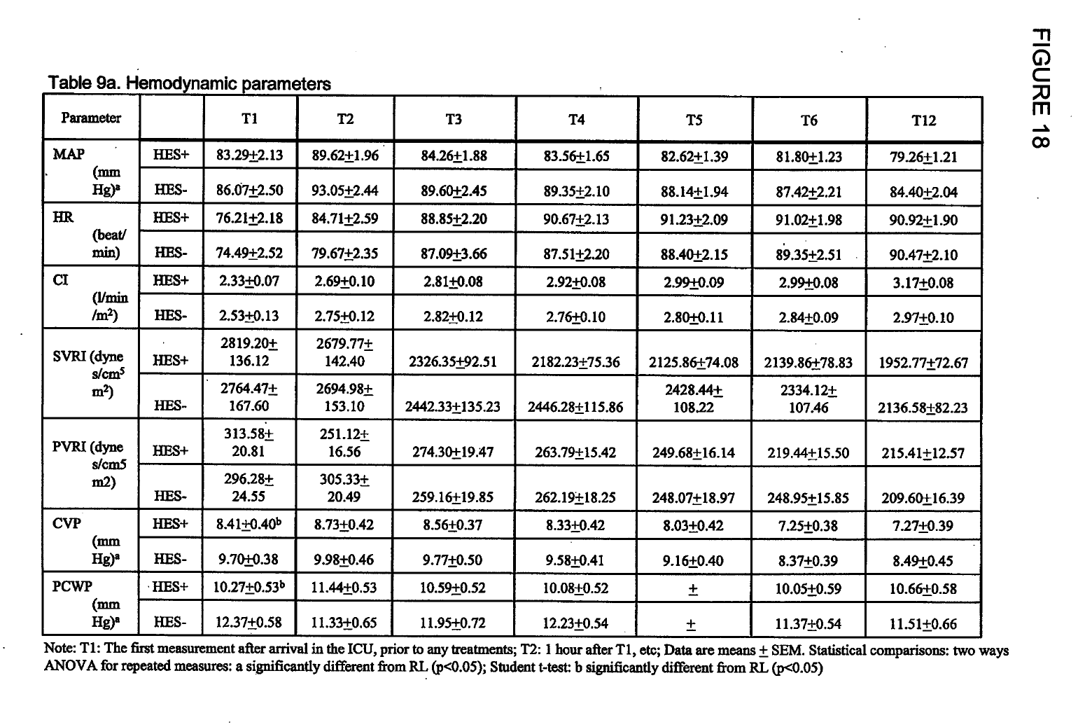

The present invention relates to a lactate and calcium containing pharmaceutical composition, to a method of preparing the pharmaceutical composition as well as to various medical and therapeutic uses of this composition. In particular, the invention relates to a pharmaceutical composition and the use thereof in the treatment of diseases and disorders such as post-operative treatment of patients, hypovolemia cardiovascular diseases, brain disorders, organ failure, obesity, acute hemodynamic distress due to medical and surgery, septic shock or obesity. In one particular aspect, the invention also relates to the use of a hypertonic lactate solution for the treatment of brain disorders.

Lactic acid as such or in form of its anion, the lactate anion or salts thereof, has found rather widespread application in the pharmaceutical field. Traditionally, the lactate anion is used as buffering agent in compositions for dialysis, see for example, Chung et al. Perit. Dial. Int. 2000, 20 Suppl. 5: S57-67, or U.S. Pat. No. 6,610,206. Lactate is also an ingredient in Ringer's lactate, an aqueous solution that is isotonic with the human blood (containing 130 mmol/l Na⁺, 5.4 mmol/l K⁺, 1.85 mmol/l Ca²⁺, 27 mmol/l lactate and 112 mmol/Cl⁻), used as physiological saline solution for intravenous infusion in hypovolemia. In addition, lactate has been described in U.S. Pat. No. 5,100,677 as one permanent mono-anionic metabolite selected from the group of pyruvate, lactate, d-betahydroxybuytrate, acetoacetate that can be employed for fluid therapy. According to this patent a solution containing 0.01 to 2400 mmol/l L-lactate is suitable for parental, oral, dialysis and irrigation therapy. Specific examples of conditions that can be treated according to this US patent are acidosis, dehydration, blood electrolyte depletion, shock, malnutrition and uremia.

More recently, the lactate anion has also been the subject of research in patients undergoing cardiac surgery. In this study, the metabolic and hemodynamic effects of a 1 M lactate solution (consisting of 90 g lactate and 23 g sodium per liter) was investigated in postoperative patients who underwent elective coronary artery bypass grafting (CABG) (Mustafa, I. and Leverve, X. M. Shock, 18, 306-310, 2002). The authors concluded from this study that this lactate solution is safe and well tolerated in patients that undergo this kind of surgery.

WO 98/08500 discloses hypertonic compositions containing L-arginine as an active ingredient besides a crystalloid for the treatment of, inter alia, traumatic brain injury. Amongst other compounds such as sodium chloride or sodium acetate, sodium lactate is disclosed in WO 98/08500 as potential crystalloid/buffer agent.

Finally, WO 2004/096204 discloses a composition containing 250 to 2400 millimoles per liter of lactic acid or lactate, 2 to 10 millimoles per liter of potassium and optionally 2 to 5 millimoles calcium per liter. According to WO 2004/096204 this composition can used for various therapeutic purposes such as therapeutic indications such as the treatment of an elevated intracranial blood pressure (ICP) or brain edema which can be caused by traumatic brain injury, the treatment of acute hemodynamic distress caused by multitrauma, shock or post-operative situations, for example.

However, despite these promising developments it would be still desirable to have a lactate containing composition that is easy to manufacture, easy to use and suitable for a great variety of therapeutic applications. Therefore, it is an object of the invention to provide such a composition.

This object is solved among others by the pharmaceutical composition having the features of the respective independent claim. Such a composition is a pharmaceutical composition containing 250 to 5000 millimoles per liter of lactic acid or lactate, and 0.5 to 1.99 millimoles per liter of calcium.

The invention is based on the finding that hypertonic lactate and calcium containing compositions (compositions that comprise lactate as active ingredient in concentrations such as described here) have highly versatile applications and a high effectiveness in therapeutic indications such as the treatment of hypovolemia and post-operative treatment such as treatment of patients that have undergone coronary artery bypass grafting (CABG) or percutaneous transluminal coronary angioplasty (PTCA), for example. The combination of the metabolisable lactate anion and the calcium ion in the concentration range as described herein, for example 0.5 to 1.99 millimoles calcium, has been found to powerfully increase the hemodynamic function of a patient. For example, the combined presence of lactate and calcium significantly increase the cardiac contraction (due to an inotropic effect). In addition, this combination allows to relax tone both in the general circulation of in the pulmonary vascularisation (decrease in vascular resistance), which results in a significant increase in cardiac output, even in patients with cardiac failure, for example. In addition, clinical studies indicate that the administration of a composition of the invention as post-operative treatment improves the neurocognitive function or status of patients that, for example, underwent cardiac surgery. The composition described here has also a remarkable anti-ischemic/antioxidant effect, and can thus be used for improving the recovery of affected patients after an ischemia-reperfusion injury. In this connection, it should further be noted that the composition of the invention also possesses a significant volume effect (fluid replenishment) rendering it an attractive agent for patients requiring fluid infusion for resuscitation, for example.

In presently preferred embodiments of the invention, the concentration of lactic acid or the lactate anion is in the range of about 350 to about 2500 mmol, or about 400 to about 1500 mmol or in the range of 500 to about 1500 millimoles per litre. In other preferred embodiments the concentration of lactic acid or the lactate anion is in the range of about 800 to about 1200 millimoles per litre. In some embodiments a concentration of the lactic acid or lactate of about 500 or about 1000 millimoles per litre has found to be particularly suitable. In this context it is noted that the term “about” when used herein with regard to the lactate concentration typically means a deviation of ±10 to ±50 mmol/liter.

However, depending on the concrete application and also on the severity of the condition and the individual to be treated, any suitable lactate concentration within the range of 250 to 5000 mmol/l, can be chosen. Accordingly, any lactate concentration within this range, for example, 350, 500, 800, 2200, 3500 or 4800 mmol lactate, can be used in combination with any concentration of the other ingredients which may be present in the composition of the invention, for example, any potassium concentration which is in the range of 2 to 10 millimoles or a calcium concentration that is within the range a described herein.

It should also be noted in this respect, that the term “lactate” comprises both enantiomeric forms, i.e. D-lactate as well as the L-lactate, wherein L-Lactate is preferred. However, as long as the D-lactate is present in amounts which do not have an adverse or even toxic effect on the patient to be treated, a mixture of L- and D-lactate can also be used in the invention. The term “lactic acid” accordingly also includes D-lactic and L-lactic acid and further includes polymeric or oligomeric forms of lactic acid such a polylactic acid (polylactate). Furthermore, derivates of lactic acid such as esters of lactic acid are also within the meaning of the term “lactic acid”. Examples of such esters are methyl lactate, ethyl lactate, or esters of lactate acid with polyols such as glycerol to name a few. Furthermore, the use of mixtures of lactic acid, lactic acid derivates such as esters thereof, and lactate is also within the scope of the invention, i.e. a pharmaceutical composition may contain lactic acid, polylactic acid and a lactate salt.

In order to achieve electroneutrality of the composition of the invention (especially, once the composition is in present as fluid), a cation such as ammonium, dimethylammonium, diethylammonium, sodium or a mixture of such cations is also present in the composition, if lactate is used. Preferably, sodium is used in some embodiments as counter-ion for the lactate anion, i.e. in those cases the concentration of sodium is identical to the chosen lactate concentration. For this reason, sodium lactate is a preferred compound used in preparing a composition of the invention. If lactic acid is used, no other cation (except protons or H₃O⁺, which result from the dissociation of lactic acid) needs to be present in order to achieve electroneutrality. However, if wanted, physiologically useful cations as described below can be present in addition to the lactic acid, if lactic acid is used as active ingredient in the present invention.

In some embodiments, the calcium concentration in the composition of the invention is in the range of about 1.2 to about 1.75 millimoles per liter, of about 1.3 to about 1.6 millimoles per liter or about 1.3 to about 1.7 millimoles per liter. In one embodiment the calcium concentration is exactly or about 1.36 millimoles per liter. In this context it is noted that the term “about” when used herein with regard to the calcium concentration typically means a deviation of ±0.1 or ±0.2 mmol/liter.

In addition, the composition may also contain potassium. The presence of potassium has been found to be in particular useful in order to prevent a hypokalemia which may be caused by the treatment with hypertonic sodium lactate alone. In some embodiments of the composition of the invention, the potassium concentration is in the range of 2 to 10 millimoles per litre potassium, or 2.5 to 6 millimoles per litre. In some embodiments a potassium concentration of about 3.5 mmol or about 4 mmol/L is presently preferred. In this context it is noted that the term “about” when used herein with regard to the potassium concentration typically means a deviation of ±0.1 to ±0.2 mmol/liter.

In addition to the above-described components, the composition according to the invention comprise one or more anions for providing electroneutrality for the calcium and optionally also the potassium that can be present in a composition of the invention. Every pharmaceutically acceptable anion can be used for this purpose. Examples of such anions include inorganic and organic anions such as chloride, iodide, phosphate, sulphate, citrate, or malonate, to name only a few. In some embodiments, the composition comprises chloride as negatively charged counter-ion for both the potassium and the calcium cation.

In accordance with the above disclosure, the composition of the invention is preferably used as an aqueous solution.

In one particular embodiment, the composition of the invention contains the above-mentioned ingredients in the following concentrations:

about 1000 millimoles per liter lactate,

about 4 millimoles per liter potassium (K),

about 1.36 millimoles per liter calcium (Ca), and

about 1000 millimoles per liter sodium (Na).

If chloride is used as counter-ion for both potassium and calcium, the chloride concentration is thus about 6.72 mol/l.

In another particular embodiment, the composition of the invention contains the above-mentioned ingredients in the following concentrations:

about 500 millimoles per liter lactate,

about 4 millimoles per liter potassium (K),

about 1.36 millimoles per liter calcium (Ca), and

about 500 millimoles per liter sodium (Na).

If chloride is used as counter-ion for both potassium and calcium in this embodiment, the chloride concentration is thus about 6.72 mol/l.

In another particular embodiments, the following concentrations are used in the composition:

about 500 millimoles per liter lactate,

about 3.5 to 4.2 millimoles per liter potassium (K),

about 1.2 to 1.4 millimoles per liter calcium (Ca), and

500 millimoles per liter sodium (Na).

If chloride is used as counter-ion for both potassium and calcium in this embodiment, the chloride concentration is thus about 4.9 to 6.8 mol/l.

Yet another example of a presently preferred embodiment is a composition having the following concentrations:

about 750 millimoles per liter lactate,

about 3.5 to 4.2 millimoles per liter potassium (K),

about 1.2 to 1.4 millimoles per liter calcium (Ca), and

750 millimoles per liter sodium (Na).

If chloride is used as counter-ion for both potassium and calcium, the chloride concentration is thus about 4.9 to 6.8 mol/l.

Yet another presently preferred embodiment is a composition having the following concentrations:
504 millimoles per liter lactate,
4.02 millimoles per liter potassium (K),
1.36 millimoles per liter calcium (Ca),
504 millimoles per liter sodium (Na).
6.74 millimoles per liter chloride (used as counter ion for K and Ca)

The composition may further contain other ingredients, for example, further physiologically relevant cations such as magnesium or zinc. Magnesium may be present in a concentration of up to about 3 or about 4 mmol/liter. The composition may also contain phosphate, in addition to such physiologically relevant cations or independent from their presence. The phosphate may be added in any suitable form, for example, as monohydrogen or dihydrogen phosphate. Examples of suitable phosphate salts are NaH₂PO₄ and Na₂HPO₄. If present, the phosphate is typically employed in a concentration up to 5 mmol/liter. A further compound that may also be added to the composition of the invention in a concentration of up to about 5 mmol/liter is ATP. ATP may be used in the form of its magnesium salt.

Other suitable additives that may be included in the composition are agents that exert an osmotic effect (osmolytes and oncotic agents) and thus can further increase the osmotic effect of the composition of the invention. Examples of such osmolytes and oncotic agents include, but are not limited to, carbohydrate compounds, gelatine, alginate, polyvinyl-pyrolidone, serum proteins such as albumin or mixtures thereof. Examples of suitable carbohydrate compounds are pectin, sorbitol, xylitol, dextrose, polydextrose, condensed glucose, modified and unmodified starch such as hydroxyethyl starch (HES), pentamethyl starch (penta starch), carboxymethyl starch or mixtures of these carbohydrate compounds. These carbohydrates may usually be present in a concentration of up to about 10% (w/v). For example, a typical concentration of hydroxyethyl starch is 6% (w/v). If another oncotic agent such as gelatine is chosen as additive, it is typically present in an amount up to about 3.5% or 4%.

As already mentioned, the composition of the invention can be used in the large variety of therapeutic applications. It may, for example, be used in the treatment of a disease or condition selected from hypovolumia (used herein in its regular meaning of designating a state of the body of decreased volume of blood plasma), coronary diseases, brain disorders, organ failure, obesity, and acute hemodynamic distress due to medical and surgery. The composition can also be used for resuscitation and also for operative/postoperative treatment of patients.

One embodiment of post-operative treatment is directed to the use of a composition of the invention for treatment or prevention of edema. The edema can be caused by or associated with any kind of treatment that a patient has received, for example, cardiac surgery, renal surgery, cosmetic surgery or orthopaedic surgery, to name only a few. It should be noted that the edema to be treated or prevented can also be independent from post operative treatment and can be associated with or caused by a condition such as burn wounds (or another condition where hypovolumia occurs of extravasation of fluid and protein), trauma, for example, traumatic brain injury, or organ failure such as (congestive) heart failure or chronic venous insufficiency.

Another embodiment of post-operative treatment is directed to the use of a composition of the invention for post-operative treatment (for example resuscitation) of patients that have undergone heart surgery. The heart surgery is typically invasive heart surgery such as open heart surgery. Examples or heart surgery after which the patients can be treated with a composition described herein include, but are not limited to, non-elective coronary artery bypass grafting (CABG) or percutaneous transluminal coronary angioplasty (PTCA), also known as angioplasty or balloon angioplasty.

The term “CABG” is used herein in its regular meaning to refer to a surgical procedure wherein a healthy blood vessel is taken from another part of the body of the patient (usually the leg or inside the chest wall) and used to construct a detour around the blocked coronary artery. In this procedure, one end of the vessel is grafted (attached) right below the blockage while the other end is grafted right above the blockage. As a result, blood can flow to the heart muscle again. The term CABG comprises also multiple bypass surgery such as double bypass surgery (wherein two grafts are performed), triple bypass, or quadruple bypass surgery.

The term “PTCA” is also used herein in its regular meaning to refer to a surgical procedure in which first a catheter is inserted and guided toward the blocked area of an effected artery and then a second catheter with a small balloon on the tip is passed through the first catheter. Once the balloon tip reaches the blocked area, the balloon is inflated. This compresses the plaque build-up, widening the artery for blood flow. Finally, the balloon is deflated and removed in PTCA.

In accordance with the above disclosure, the composition of the invention can thus also be used in emergency cases (for example, for the treatment of an increased intracranial pressure as discussed in detail below), as an agent in intensive care units (ICU) as well as parenteral food supplement for obese or hypercatabolic patients.

One therapeutic application of interest is the use of the pharmaceutical composition of the invention for the treatment of a brain disorder. Also in this case, only lactate and/or lactic acid and calcium need to be present in a composition of the invention. Examples of such brain disorders are traumatic brain injury, cerebral ischemia or non-traumatic brain injury, metabolic disorders associated with brain dysfunction and complications associated with surgery.

In one embodiment the traumatic brain injury is closed or open craniocerebral trauma (CCT). To the surprise of the inventors, it was found that the pharmaceutical composition of the invention is not only able to significantly reduce an increase in the intracranial pressure (ICP) which is caused by the traumatic brain injury but that the efficiency exceeds that of mannitol, which is the standard osmotherapeutic approach for lowering an increased ICP.

In addition, the composition of the invention can also be administered to a patient that suffers from a non-traumatic brain injury such as stroke or cold-lesion or to a patient having a metabolic disorder associated with brain dysfunction such as hepatic or hypoglycemic coma. Due to its strong osmotic effect, the composition of the invention is also useful for the treatment of any (intracellular) brain edema caused by a traumatic or non-traumatic brain injury (disorder) so this edema is either reduced or prevented.

Examples of cardiovascular diseases or coronary diseases that can be treated with the composition of the invention are myocardial ischemia, cardiac dysfunction, cardiac and vascular complications of diabetes, acute infarction, ischemic reperfusion injury, or complications of arteriosclerosis to name a few.

As the composition generally exerts an anti-ischemic effect, it can also be used in the treatment of a patient suffering from the failure of any organ. Examples of specific organ failures that can be treated include, but are not limited to renal failure, liver failure or heart failure. In addition, it is for example also possible to treat cardiogenic shock which is caused by heart failure with the composition of the invention.

The composition of the invention has also been found to be useful for the treatment of any form of acute hemodynamic distress. This acute stress may, for example, be caused by polytrauma, post-operative situations, septic shock, respiratory diseases, or acute respiratory distress syndrome.

In accordance with the above disclosure, a composition disclosed here is usually administered as a fluid. For this purpose, any suitable way of administering a fluid to a patient can be used. Preferably, the composition is administered parenterally by infusion or injection (for example by intravenous, intramuscular or intracutaneous administration). For intravenous administration the composition of the invention may be given as continuous infusion, bolus infusion or bolus injection, for example. A typical daily maximum dosage of lactate is about 4.5 to about 7.5 mmol/kg body weight/day or about 4.5 to about 10 mmol/kg body weight/day, or calculated with a body weight of 70 kg 0.315 to 0.525 mol lactate/day or 0.315 mol to 0.700 mol lactate/day. The amount that is considered suitable for a patient can be given administered in any suitable dosage. For example, using a composition that contains about 500 mM lactate, an amount of up to 5 mmol/kg (corresponding to 10 ml/kg body weight of a 0.5 m lactate solution or a total volume of 700 ml for a patient with a body weight of 70 kg) can be continuously infused in up to 12 hours. Such a dosing regime may for example, be chosen for post-operative treatment of patients after cardiac surgery. Alternatively, if a solution with a lactate concentration of 2500 mmol/liter is used, an amount of 5 mmol lactate/kg body weight can be administered by continuous infusion within about 2.4 hours (the total infused volume is 140 ml for a patient with a body weight of 70 kg). If wished, the amount of 5 mmol lactate/kg body weight could also be administered by bolus injection using a solution with a lactate concentration of 5000 mmol/liter. In this case, for example, 5 bolus injection of each 14 ml of such a lactate solution could be given to a patient over a time period of 12 hours. If polylactate is employed in the composition of the invention, oral administration is a preferred route.

The invention further relates to a method of preparing a pharmaceutical composition containing 250 to 5000 millimoles per liter of lactic acid or the lactate, 0.5 to 1.99 millimoles per liter of calcium and optionally, if present, also 2 to 10 millimoles per liter of potassium. This method comprises in one preferred embodiment providing respective amounts of sodium lactate or lactic acid, calcium chloride and optionally, potassium chloride and dissolving the compounds in a pharmaceutically acceptable solvent. In this respect, it is noted that the ingredients necessary for the preparation of a liquid composition of the invention, for example, sodium lactate, lactic acid, calcium chloride and potassium chloride can also be mixed as solids and this mixture is then dissolved in a pharmaceutically acceptable solvent only prior to its administration to a patient in need thereof. Accordingly, a pharmaceutical composition comprising lactate or lactic acid, and calcium (and optionally also any additional ingredients such as potassium or magnesium or an osmolytic agent) in solid form is also within the scope of the present invention. In some circumstances, for example, if storage room is limited, it might even be of advantage to prepare a solid mixture of the components of the composition of the invention, and prepare a liquid form thereof, only when needed.

In principle, every suitable combination of compounds that yield a composition having the desired content can be used for preparing the composition of the invention. For example, a composition can be prepared from lactic acid, sodium lactate, calcium chloride (×2 H₂O), and optionally potassium chloride. Alternatively, a mixture of calcium lactate, sodium lactate, and optionally sodium chloride could also be used for preparing a composition of the present invention.

The solvent can be any suitable pharmaceutical acceptable solvent, for example, water, or a mixture of water with an organic solvent such as ethanol, as long as this solvent is able to dissolve the solid components, in particular of the composition in the specified amounts. Typically, the solvent is deionised, single or double distilled or micro-filtered water the purity of which is acceptable for pharmaceutical applications. The fluid composition so prepared can be treated further, for example, by heat sterilisation or sterile-filtration before administered to a patient. An example of a preferred solvent/pharmaceutical carrier used for the preparation of the composition of the invention is sterile Water for Injection (WFI) as classified by the United States Pharmacopiea (USP).

The invention is further illustrated by the attached Figures and the following non-limiting Examples.

### EXAMPLE

### Efficacy and Safety of Hypertonic Lactate Solution as Fluid Resuscitation Compared with Modified Ringer's Lactate in Post-CABG (Coronary Artery Bypass Grafting) Patients

A randomized, open-label study was carried out in order to assess the efficacy and safety of a hypertonic lactate calcium containing solution of the invention (HL) compared to a Ringer's Lactate (RL) as fluid resuscitation to maintain haemodynamic stability in post-CABG patients. The composition of the used Ringer's Lactate is given below.

Post-CABG patients aged 18-75 years in intensive care unit (ICU) who were in need of fluid resuscitation were included in this study. 230 patients were enrolled for this purpose: 208 patients were analyzed, 109 patients from the HL group and 99 patients from the RL group; 22 patients were withdrawn due to protocol violation, 6 patients from the HL group and 16 from the RL group. The demographics and baseline characteristics of the patients are summarized in Table 1.

Patients who were eligible received hypertonic lactate solution to a maximal dose of 10 ml/kg body weight or Ringer's Lactate to a maximal dose of 30 ml/kg body weight over the first 12 hours post-CABG in the intensive care unit (ICU) when fluid resuscitation was needed. In the HL group of the study, it was allowed to administer hydroxyethylstarch (HES) if more fluid required and the maximum dose of hypertonic lactate solution has been reached.

Patients were excluded if patients underwent combined operations, required intra aortic balloon pump or patients with severe arrhythmia (VT, AF rapid response, heart block), severe haemodynamic balance, severe bleeding or re-operation. Patients with hypernatremia (Na>155 mmol/L), severe liver failure (SGOT and SGPT>2× normal value), or severe renal failure (creatinine>2 mg %) were also excluded.

The hypertonic lactate solution according to the invention used in this trial was a solution with an osmolarity value of 1020 mOsm/L in a clear colorless glass bottle and with the following composition:

| Ingredient | Amount/1 000 mL | | Concentration (mmol/l) |
|---|---|---|---|
| Sodium lactate sol. | 113 g (equivalent | | 504 |
| (50% w/v) | to 56.5 g g) | | |
| Potassium Chloride | 0.30 | g | 4.02 |
| Calcium Chloride × | 0.20 | g | 1.36 |
| 2H₂O | | | |
| Water for injection | Ad 1 000 | ml | |

This hypertonic lactate solution was administered intravenously through a central vein to a maximal volume of 10 ml/kg body weight over the first 12 hours.

A ready to use Ringer's Lactate solution in plastic bottle of the following composition was used as comparison:

| Ingredient | Amount/1000 mL | | Concentration (mmol/l) |
|---|---|---|---|
| Sodium lactate | 3.1 | g | 25.4 |
| Sodium chloride | 6.0 | g | 102 |
| Potassium Chloride | 0.30 | g | 4.02 |
| Calcium Chloride | 0.20 | g | 1.80 |
| Water for injection | Ad 1000 | ml | |

The Ringer's lactate was administered intravenously to a maximal dose of 30 ml/kg body weight over the first 12 hours.

A ready to use hydroxyethylstarch (HES) solution in plastic bottle of the following composition was used when the maximum dose of hypertonic sodium lactate was reached.

| Ingredient | Amount/1000 mL | | Concentration (mmol/l) |
|---|---|---|---|
| O-(2-hdroxyethyl)- | 60 | g | |
| amylopectin hydrolysate | | | |
| HES MW 200,000 | | | |
| Sodium lactate (50% | 4.48% | | |
| w/v) | | | |
| Sodium chloride | 6.0 | g | 102 |
| Potassium Chloride | 0.30 | g | 4.02 |
| Calcium Chloride × 2H₂O | 0.22 | g | 1.50 |
| Water for injection | Ad 1000 | ml | |

The efficacy of the solutions used in the study was assessed by:

1. Hemodynamic status (Cardiac Index (CI), Mean Arterial Pressure (MAP), Pulmonary Vascular Resistance Index (PVRI), Systemic Vascular Resistance Index (SVRI), Central Venous Pressure CVP, Pulmonary Capillary Wedge Pressure (PCWP), Heart rate (HR)
2. Body fluid balance (urinary output; total fluid losses including urine, drainage and hemorrhage; total fluid infusion including hypertonic lactate solution or modified Ringers Lactate, blood product and other fluids).
3. Reduced concomitant ionotropic drug utilization.

The safety was assessed by: 1. the laboratory parameters hemoglobin, hematocrit, sodium, potassium, chloride, calcium, magnesium, lactate, and Blood Gas Analysis (pH, PO₂, PCO₂, bicarbonate). 2. Any clinical signs which the investigator considers significant.

Statistical Methods Assessment of comparability between HL group and RL group by either unpaired student t-test or Chi-Square test or by a two-way ANOVA for repeated measures followed by post-hoc analysis when a significant difference was found within the two groups (Statview).

### Detailed Description of Study Set-Up:

After surgery, patients were observed in the ICU. During this immediate post-operative period, patients were filled to maintain the PCWP between 12-15 mmHg and/or CVP between 8-12 mmHg by using either Ringers Lactate or Hypertonic lactate solution according to the group to which the patient was assigned. Treatment was administered according to the body weight, hypertonic lactate solution was given in HL group to a maximum dose of 10 ml/kg BW for up to 12 hours post CABG and Ringer's Lactate was given in RL group to a maximum dose of 30 ml/kg BW for a similar duration. When the maximum dose of hypertonic lactate solution was reached, it was permitted to infuse HES in case of necessity of maintaining fluid therapy.

Post-operative care was standardized: mean arterial pressure was maintained between 60-90 mmHg with either dopamine or norepinephrine and milrinone or nitroglycerine (NTG) when necessary. Hemoglobin concentration was maintained around 10 mg/dl, with blood transfusion when needed. Cardiac index and other hemodynamic parameters were maintained with inotropic agents, vasodilators and/or fluid resuscitation, taking into account the overall haemodynamic balance of the patients and the specific effect(s) of the drugs. For instance, if target PCWP and CVP were reached but CI was still below 2.5 l/min/m² on the presence of low Systemic Vascular Resistance (SVR), Norepinephrine was given. However if SVR was high, milrinone was given; and if SVR was normal, dobutamin was given.

Hemodynamic parameters including heart rate (HR), systolic, diastolic and mean arterial pressure (MAP), cardiac output, vascular resistance, central venous pressure (CVP) and pulmonary capillary wedge pressure (PCWP) were assessed when patients arrived in the ICU and monitored every hour for the immediate 6 h afterwards and then at the 12^{th} h. Parameters such as cardiac index (CI), systemic vascular resistance index (SVRI) and pulmonary vascular resistance index (PVRI) were subsequently calculated using standard formulae. In this regard it is noted that due to the nature of patient management in ICU where patient stabilization is of primary importance, it was not possible to obtain hemodynamic parameters immediately upon arrival in ICU and before the administration of any fluid as baseline values. The earliest measurement could be done 1 hour upon arrival. Within this 1 hour time, some of the patients needed to be already given fluid (51 in the HL group and 48 in the RL group), hence in these patients measurement at T1 (hour) could not be regarded as baseline. In the remaining patients (58 in the HL group and 51 in the RL Group), T1 could be regarded as baseline values as described in Table 2b.

Several other relevant biological parameters were determined when patients come into the ICU and then 6 and 12 hours afterwards using blood drawn from either the arterial (PaO₂, PaCO₂, pH and bicarbonate) or the venous (Na⁺, K⁺, Cl′, Ca⁺⁺, Na⁺, Mg⁺⁺, Lactate) lines. The hemoglobin (Hb) and hematocrit (Ht) values at these hours were also measured. Total urine and bleeding were measured hourly.

### Efficacy Results

### Hemodynamic Effects.

There was no significant difference in baseline hemodynamic parameters except for PVRI (p<0.05). Changes in all hemodynamic parameters of the whole cohort of patients were observed over the immediate 12 hr postoperative period in the ICU as follows: MAP, SVRI, PVRI, CVP and PCWP significantly decreased (p<0.0001) while HR following CI significantly increased (p<0.0001). The clinical objectives for fluid resuscitation as well as for inotrope/vasodilator administration were reached in both RL and HL groups. Despite similar cardiac filling pressures (CVP and PCWP) (Table 7 and Table 8), arterial pressure (Table 2a, b, c with Table 2b showing that significant differences were observed in all of hemodynamic parameters observed, which implies that baseline values for all hemodynamic parameters are the same in HL and RL groups) and HR (Table 3) within the two groups, it was however seen that the CI was significantly higher (p=0.018) in HL group as compared to RL (Table 4). Since the calcium concentrations were similar in both the composition of the invention and the Ringer's lactate, the increase in the cardiac index must be attributed to an (unexpected) “synergistic” effect of the hypertonic lactate concentration and the calcium concentration used in this solution of the present invention.

Since several patients from HL group received HES infusion, conversely to RL group, the hemodynamic status of the subgroup of patients according to HES infusion was analysed. From the comparison between HES+ and HES− it is evident that CVP (Table 14) and PCWP (Table 15) were different, both parameters being significantly lower in the HES+ group as compared to HES− since baseline (p=0.006 and p=0.025 respectively). This indicates that the cardiac filling pressures were lower in the group of patients where it seemed to be necessary to give more fluid after the maximal load of hypertonic lactate solution allowed was reached (HES+) beside the significantly lower MAP in this group (even both groups were within the acceptable range) (Table 9a, 9b), however hemodynamic status as assessed by CI (Table 11) and HR (Table 10) was identical in these two groups. Hence, these patients received more fluid, as HES, mainly because of low CVP and PCWP and not because of inadequate cardiac function. Moreover, the finding of similar PVRI (Table 13), regardless additional infusion of HES indicates that the lower resistance observed in HL group is the consequence of hypertonic lactate solution infusion rather than the use of HES.

### Body Fluid Balance

The parameters involved in body fluid balance in RL and HL: urinary output, total fluid losses (urine, drainage and hemorrhage), total fluid infusion (Ringer's lactate or hypertonic lactate solution, blood products and HES when used), and total fluid balance (total fluid infusion minus total fluid losses). Urinary output (Table 16) and total fluid losses (Table 17) over these 12 first postoperative hours were not significantly different (p>0.05) in both groups while total fluid infusion (Table 18) was markedly lower (p<0.0001) in HL as compared to RL since it was almost half (1319.70±71.30 vs. 2430.35±122.61 ml/12 h for HL and RL respectively, p<0.0001) resulting in a significant negative fluid balance (−793.40±71.37 ml/12 h, p<0.0001 versus 0), contrasting with the null fluid balance observed in the RL group (+42.71±114.73 ml/12 h, NS versus 0). Hence similar hemodynamic status and diuretic effect but with higher cardiac index were achieved in HL group as compared to RL despite a much lower fluid infusion rate and a substantial negative fluid balance, thereby demonstrating another advantage of the composition of the present invention

As already mentioned, HL group is not homogenous since some patients received an additional infusion of HES whereas others did not. Therefore it was analysed whether the fluid balance parameters according to the subgroups are linked to HES infusion or not. Total output (Table 21) was the same regardless HES infusion (p>0.05) and urinary output (Table 22) was slightly, although significantly, higher in HES+ as compared to HES− group (p=0.040). Total fluid infusion (Table 23) was significantly higher in HES+ compared to HES− (1578.77+75.09 vs 764.57+91.32 ml/12 h respectively, p<0.0001). As a consequence, body fluid balance (Table 24) was less negative in HES+ compared to HES− (−646.65+83.62 vs −1107.86+116.07 ml/12 h respectively, p<0.05).

### Concomitant Drug Utilization

Post-operative care was carefully standardized to maintain mean arterial pressure between 60 and 90 mm Hg with either dopamine or norepinephrine and milrinone or nitroglycerine when necessary. No patient required adrenaline administration. Comparison between HL and RL group showed no significant difference regarding the number of patients receiving dobutamine, nitroglycerine and norepinephrine respectively. However, milirinone was significantly less frequently used in HL group than RL group (28 versus 39%, p=0.05. Accordingly, a composition of the invention can also be used in order to reduce the administration of inotropic drugs such as milirinone in post-operative treatment of patients.

### Special Analysis to Evaluate the Effect on Cardiac Index

Measurement of cardiac index (CI) like other haemo-dynamic parameters were done hourly beginning the 1^{St} hour in the ICU (T1, T2 etc). In 98 patients (48 patients in the RL group and 50 patients in the HL group), fluids were administered within the first hour in the ICU, and hence in this group of patients no baseline data could be obtained before fluid administration. In the other 109 patients (51 given RL and 58 given HL) here fluid was administered after 1 hour, measurements at T1 could be regarded as baseline values. Presence of baseline values is important especially to see the magnitude of increase of cardiac index by fluid administration.

From Table 25 it is evident that both groups have the same baseline values. This observation implies that randomization in this study (involving a large group of patients) were effective and support the conclusions of efficacy and safety parameters described elsewhere in this report. In this group, measurement of cardiac index in subsequent hours is described in Table 26. Two way Analysis of Variance (Anova) with repeated measures gave a p value of 0.447 while one way analysis gave values as shown in Table 27. Although 2-way Anova for repeated measures shows a non-significant p value, from one way Anova analysis it was clear that there is a consistent tendency that patients receiving HL always had a higher CI compared to RL. At hour 12, the difference almost reach statistical significance (p=0.06). This reduction of statistical significance compared to the overall analysis of 208 patients shown earlier is the impact of smaller sample size due to segmentation. Table 28 depicts the increase of cardiac indices compared to baseline values in HL group while Table 29 depicts the same for RL groups. Both groups showed statistically significant increases compared to respective baseline values, however the increase in HL group (between 0.3 to 0.8) was higher compared to RL group (0.14-0.53). The difference in terms of cardiac index improvement between HL and RL group was then analysed by one-way Anova and this gives a statistically significant value (p=0.05 at hour 12) as seen in Table 30.

The efficacy results can be summarized as follows:

a) Hemodynamic function (MAP, HR, CVP, PCWP) were maintained at comparable levels between the two groups, while urine output was found to be similar between both groups. These indicated that similar tissue perfusions in the HL group could be maintained at the same level as RL group despite a much lower fluid infusion (p<0.0001) in HL group. This effect was proven to be independent of HES administration. The tendency toward a higher increase of the CI in HL group, with a lower vascular resistance compared to RL group, without a drop in MAP in addition attests for an inotropic effect.

b) Patients in HL group exhibited higher CI (p=0.0179) compared to RL group. This effect was also proven to be independent of HES administration.

c) Concomitant drug utilization: number of patients that received milrinone in HL group was significantly lower compared to RL group (28% vs. 39%, p<0.05). Less number of patients receiving milrinone is one advantage not only from a cost point of view but more importantly benefit advantage.

d) A separate analysis on CI conducted on patients whose fluid administration (HL or RL) was given after more than 1 hour in ICU, and hence their CI at T1 (hour 1) could serve as baseline values (HL group=58, and RL group=51 patients), found that the increase in cardiac index in HL group at hour 12 (0.79±0.62) was higher compared to RL (0.53±0.62); p=0.05. The facts that baseline values in term of CI in these groups were similar, and that the increase of CI observed in the group without baseline at hour 1 was already significant (2.47±0.71 versus 2.11±0.61; p=0.007) indicate that the effect of CI increase in the HL group was immediate.

Based on the above efficacy results, with comparable hemodynamic parameters and tissue perfusion, and with lower vascular resistance, patients in HL group had higher cardiac index and less total fluid infusion. The latter point is also worth noting since the infusion of less fluid significantly reduces the risk of having post-operative edematous state. In addition, an ongoing separate study with a group of patients seems to indicate that post-operative treatment of patients that have undergone cardiac surgery provides an improvement of the neurocognitive functions within about 6 months after the surgery compared to patients that were infused with the above-mentioned Ringer's Lactate.

1.-34. (canceled)

35. A pharmaceutical composition containing 250 to 5000 millimoles per liter of lactic acid or lactate, 1.2 to 1.7 millimoles per liter of calcium, and 2.5 to 6 millimoles per liter potassium.

36. The composition of claim 35, wherein the concentration of lactic acid or lactate is in the range of 400 to 2400 millimoles per liter.

37. The composition of claim 35, wherein the concentration of lactic acid or lactate is in the range of 800 to 1200 millimoles per liter.

38. The composition of claim 35, wherein the concentration of lactic acid or lactate is about 500 millimoles per liter or about 1000 millimoles per liter.

39. The composition of claim 35, wherein sodium (Na) is used as counter-ion for the lactate.

40. The composition of claim 35, wherein the calcium concentration is in the range of 1.3 to 1.5 millimoles per liter.

41. The composition of claim 35, wherein chloride (Cl) is present as counter-ion for potassium and calcium.

42. The composition of claim 35, wherein lactate is L-lactate.

43. The composition of claim 35, wherein the composition is an aqueous solution.

44. The composition of claim 39 having the concentrations:
about 1000 millimoles per liter lactate,
about 4 millimoles per liter potassium (K),
about 1.36 millimoles per liter calcium (Ca), and
about 1000 millimoles per liter sodium (Na).

45. The composition of claim 39 having the concentrations:
about 500 millimoles per liter lactate,
about 4 millimoles per liter potassium (K),
about 1.36 millimoles per liter calcium (Ca), and
about 500 millimoles per liter sodium (Na).

46. The composition of claim 35 further comprising an osmolyte selected from the group consisting of a carbohydrate compound, gelatine, alginate, polyvinyl-pyrolidone, a serum protein and mixtures thereof.

47. The composition of claim 46, wherein the carbohydrate compound is pectin, dextrose, polydextrose, hydroxyethyl starch, pentamethyl starch, carboxymethyl starch, condensed glucose, sorbitol, xylitol or a mixture thereof.

48. A method of treating or preventing a disease or condition selected from the group consisting of hypovolemia, operative treatment of patients, post-operative treatments of patients, cardiovascular diseases, brain disorders, organ failure, obesity, resuscitation, edema and acute hemodynamic distress due to medical treatment and surgery, said method comprising administering to a patient a pharmaceutical composition containing 250 to 5000 millimoles per liter of lactic acid or lactate, 1.2 to 1.7 millimoles per liter of calcium, and 2.5 to 6 millimoles per liter potassium.

49. The method of claim 48, wherein post-operative treatment of patients is treatment of patients that underwent heart surgery or prevention or treatment of edema.

50. The method of claim 49, wherein the heart surgery is coronary artery bypass grafting (CABG) or percutaneous transluminal coronary angioplasty (PTCA).

51. The method of claim 48, wherein the brain disorder is selected from the group consisting of traumatic brain injury, cerebral ischemia or non-traumatic brain injury, metabolic disorders associated with brain dysfunction and complications associated with surgery.

52. The method of claim 51, wherein the traumatic brain injury is closed or open craniocerebral trauma.

53. The method of claim 51, wherein an increased intracranial pressure caused by the traumatic brain injury is decreased.

54. The method of claim 51, wherein the non-traumatic brain injury is stroke or cold-lesion.

55. The method of claim 48, wherein the metabolic disorder associated with brain dysfunction is hepatic or hypoglycemic coma.

56. The method of claim 48, wherein brain edema caused by the brain disorder is reduced or prevented.

57. The method of claim 48, wherein the cardiovascular disease is selected from the group consisting of myocardial ischemia, cardiac dysfunction, cardiac and vascular complications of diabetes, acute infarction, ischemic reperfusion injury, and complications of arteriosclerosis.

58. The method of claim 48, wherein the organ failure is renal failure, liver failure or heart failure.

59. The method of claim 58, wherein the heart failure causes cardiogenic shock.

60. The method of claim 48, wherein the acute hemodynamic distress is caused by polytrauma, post-operative situations, septic shock, respiratory diseases, or acute respiratory distress syndrome.

61. The method of claim 48, wherein the composition is administered by infusion or injection.
